(19) United States
(12) Patent Application Publication
(10) Pub. No.: US 2005/0118579 A1
(43) Pub. Date: Jun. 2, 2005

(54) Chemical compounds

(76) Inventors: Carl John Cresswell, Macclesfield (GB); Adam Jeston Dudley, Wilmington, DE (US)

Correspondence Address: FISH & RICHARDSON PC, 225 FRANKLIN ST, BOSTON, MA, 02110, US

(21) Appl. No.: 10/468,326

(22) PCT Filed: Feb. 12, 2002
(86) PCT No.: PCT/GB02/00651

Related U.S. Application Data
(60) Provisional application No. 60/275,106, filed on Mar. 13, 2001.

(30) Foreign Application Priority Data
Feb. 17, 2001 (GB) 0103990.8

(51) Int. Cl. C12Q 1/68; C07H 21/04

(52) U.S. Cl. 435/6; 536/23.5

(57) ABSTRACT

This invention relates to polymorphisms in the human cMOAT gene and corresponding novel allelic polypeptides encoded thereby. The invention also relates to methods and materials for analysing allelic variation in the cMOAT gene, and to the use of cMOAT polymorphism in treatment of diseases with cMOAT transportable drugs.

This invention relates to polymorphisms in the human cMOAT gene and corresponding novel allelic polypeptides encoded thereby. The invention also relates to methods and materials for analysing allelic variation in the cMOAT gene, and to the use of cMOAT polymorphism in treatment of diseases with cMOAT transportable drugs.

The liver converts endogenous and xenobiotic lipophilic compounds into anionic conjugates with glutathione, glucuronate, or sulphate. These conjugates are transported across the canalicular (apical) membrane into bile by a 190 kDa membrane glycoprotein. This apical conjugate-transporting ATPase has been termed canalicular multidrug resistance protein (cMRP) because of the similarity in substrate specificity and sequence with the multidrug resistance protein (MRP1); other names include canalicular multispecific organic anion transporter (cMOAT) and multidrug resistance protein 2 (MRP2). The term “cMOAT” will generally be used herein. The amino acid sequence identity of human cMOAT and MRP1 is 49%. cMOAT is predominantly expressed in hepatocytes and localised to apical membrane domains. cMOAT is not expressed in the human Dubin-Johnson syndrome, which is therefore associated with an inherited deficiency in the secretion of amphiphilic anionic conjugates into the bile. The rat homolog CMOAT is absent in two mutant strains of rats with different point mutations in the corresponding gene. These mutant rats are hyperbilirubinemic and deficient in the ATP-dependent transport of conjugates from hepatocytes into bile. Impairment of bile flow (cholestasis) can be associated with a down-regulation of the expression of the conjugate export pump, and cMOAT contributes to bile flow as an important driving force (for reviews see Keppler D. and Konig J. FASEB J, 1997. 11, 509-516 and Ishikawa et al. Biosci Rep 1997; 17:189-207).

cMOAT has also been shown to be involved in the transport of xenobiotics, and drugs involved in lipid lowering e.g. statins. Statins have been refered to as a first-line therapy for patients with atherosclerotic vascular diseases. The cMOAT gene and its product is also thought to be of importance in other diseases.

All positions herein of polymorphisms in the exon regions of the cMOAT polynucleotide relate to the position in SEQ ID NO 1 unless stated otherwise or apparent from the context. At the time of writing, EMBL U49248 is equivalent.

All positions herein of polymorphisms in the 5′ UTR or promoter region of the cMOAT polynucleotide relate to the position in SEQ ID NO 2 unless stated otherwise or apparent from the context. At the time of writing, EMBL AF1446309 is equivalent.

All positions herein of polymorphisms in the intron regions of the cMOAT polynucleotide relate to the position in SEQ ID NO 3, 4, 5 or 6 depending on the context used. At the time of writing, SEQ ID NO 3, is part of a bacterial artificial chromosome RP11-483F11 (Sanger Centre, Hinxton, Cambridgeshire, CB10 1SA, UK.); EMBL AL133353 wherein SEQ ID NO 3 position 1=position 129361 in published BAC sequence acc no AL133353.

All positions herein of polymorphisms in the cMOAT polypeptide relate to the position in SEQ ID NO 7 unless stated otherwise or apparent from the context.

One approach is to use knowledge of polymorphisms to help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Clinical trials have shown that patient response to treatment with pharmaceuticals is often heterogeneous. Thus there is a need for improved approaches to pharmaceutical agent design and therapy.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The present invention is based on the discovery of polymorphisms in cMOAT. In particular, we have found eight polymorphisms in the coding sequence of the cMOAT gene, 6 of which lead to changes in the sequence of expressed protein; three polymorphisms in the 5′UTR; and seven intronic polymorphisms.

According to one aspect of the present invention there is provided a method for the diagnosis of a polymorphism in cMOAT in a human, which method comprises determining the sequence of the human at at least one polymorphic position and determining the status of the human by reference to polymorphism in cMOAT. Preferred polymorphic positions are one or more of the following positions:
positions 78, 1350, 1584, 1686, 2647, 3208, 3664 and 4391 in the coding region of the cMOAT gene as defined by the position in SEQ ID NO: 1;
positions 1349, 1875 and 1879 in the 5′UTR region of the cMOAT gene as defined by the position in SEQ ID NO: 2;
positions 12704 and 29446 in the intron region of the cMOAT gene as defined by the position in SEQ ID NO: 3;
position 292 in the intron region of the cMOAT gene as defined by the position in SEQ ID NO: 4;
positions 232 and 457 in the intron region of the cMOAT gene as defined by the position in SEQ ID NO: 5;
positions 50 and 68 in the intron region of the cMOAT gene as defined by the position in SEQ ID NO: 6; and
positions 417, 495, 529, 849, 1036 and 1188 in the cMOAT polypeptide as defined by the position in SEQ ID NO: 7.

The term human includes both a human having or suspected of having a cMOAT mediated disease and an asymptomatic human who may be tested for predisposition or susceptibility to such disease. At each position the human may be homozygous for an allele or the human may be a heterozygote.

The term polymorphism includes single nucleotide substitution, nucleotide insertion and nucleotide deletion which in the case of insertion and deletion includes insertion or deletion of one or more nucleotides at a position of a gene and corresponding alterations in expressed protein.

In one embodiment of the invention preferably the method for diagnosis described herein is one in which the polymorphism in the coding region of the cMOAT gene as defined by the position in SEQ ID NO: 1 is any one of the following:

| Position | DNA |
|---|---|
| SEQ ID NO 1 | Variation |
| 78 | C/T |
| 1350 | G/A |
| 1584 | A/G |
| 1686 | C/T |
| 2647 | T/G |
| 3208 | T/C |
| 3664 | A/T |
| 4391 | T/G |

In one embodiment of the invention preferably the method for diagnosis described herein is one in which the polymorphism in the in the 5′UTR region of the cMOAT gene as defined by the position in SEQ ID NO: 2 is any one of the following:

| Position | DNA |
|---|---|
| SEQ ID NO 2 | polymorphism |
| 1349 | G/A |
| 1875 | G/A |
| 1879 | A/G |

In one embodiment of the invention preferably the method for diagnosis described herein is one in which the polymorphism in the intronic regions of the cMOAT gene is any following:

| Position | DNA polymorphism |
|---|---|
| 12704 SEQ ID NO 3 | C/T |
| 29446 SEQ ID NO 3 | C/T |
| 292 SEQ ID NO 4 | T/C |
| 232 SEQ ID NO 5 | C/G |
| 457 SEQ ID NO 5 | A/G |
| 50 SEQ ID NO 6 | G/A |
| 68 SEQ ID NO 6 | G/A |

In one embodiment of the invention preferably the method for diagnosis described herein is one in which the polymorphism in the cMOAT protein is any one of the following in SEQ ID NO 7: Val417Ile, Lys495Glu, Arg529Trp, Leu849Arg, Ile1036Thr and Glu1188Val.

The method for diagnosis is preferably one in which the sequence is determined by a method selected from amplification refractory mutation system, restriction fragment length polymorphism and primer extension.

The status of the individual may be determined by reference to allelic variation at any 1, 2, 3, 4, 5, 6, 7, 8, 9, 10, 11, 12, 13, 14, 15, 16, 17, 18 or more positions.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| ELISA | Enzyme Linked ImmunoSorbent Assay |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

Table 1—Mutation Detection Techniques
General: DNA sequencing, Sequencing by hybridisation
Scanning: PTT*, SSCP, DGGE, TGGE, Cleavase, Heteroduplex analysis, CMC, Enzymatic mismatch cleavage
* Note: not useful for detection of promoter polymorphisms.
Hybridisation Based: Solid phase hybridisation: Dot blots, MASDA, Reverse dot blots, Oligonucleotide arrays (DNA Chips).
Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)
Extension Based: ARMS™, ALEX™—European Patent No. EP 332435 B1 (Zeneca Limited), COPS—Gibbs et al (1989), Nucleic Acids Research, 17, 2347.
Incorporation Based: Mini-sequencing, APEX
Restriction Enzyme Based: RFLP, Restriction site generating PCR
Ligation Based: OLA
Other: Invader assay
   Table 2—Signal Generation or Detection Systems
Fluorescence: FRET, Fluorescence quenching, Fluorescence polarisation—United Kingdom Patent No. 2228998 (Zeneca Limited)
Other: Chemiluminescence, Electrochemiluminescence, Raman, Radioactivity, Colorimetric, Hybridisation protection assay, Mass spectrometry
   Table 3—Further Amplification Methods
SSR, NASBA, LCR, SDA, b-DNA
   Table 4—Protein Variation Detection Methods
Immunoassay
Immunohistology
Peptide sequencing

Preferred mutation detection techniques include ARMS™, ALEX™, COPS, Taqman, Molecular Beacons, RFLP, and restriction site based PCR and FRET techniques. Immunoassay techniques are known in the art e.g. A Practical Guide to ELISA by D M Kemeny, Pergamon Press 1991; Principles and Practice of Immunoassay, 2^{nd} edition, C P Price & D J Newman, 1997, published by Stockton Press in USA & Canada and by Macmillan Reference in the United Kingdom.

Particularly preferred methods include ARMS™ and RFLP based methods. ARMS™ is an especially preferred method.

In a further aspect, the diagnostic methods of the invention are used to assess the pharmacogenetics of a drug transported by cMOAT.

Assays, for example reporter-based assays, may be devised to detect whether one or more of the above polymorphisms affect transcription levels and/or message stability.

Individuals who carry particular allelic variants of the cMOAT gene may therefore exhibit differences in their ability to regulate protein biosynthesis under different physiological conditions and will display altered abilities to react to different diseases. In addition, differences arising as a result of allelic variation may have a direct effect on the response of an individual to drug therapy. The diagnostic methods of the invention may be useful both to predict the clinical response to such agents and to determine therapeutic dose.

In a further aspect, the diagnostic methods of the invention, are used to assess the predisposition and/or susceptibility of an individual to diseases mediated by cMOAT. This may be particularly relevant in the development of hyperlipoproteinemia and cardiovascular disease and the present invention may be used to recognise individuals who are particularly at risk from developing these conditions.

In a further aspect, the diagnostic methods of the invention are used in the development of new drug therapies which selectively target one or more allelic variants of the cMOAT gene. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention the presence or absence of variant nucleotides is detected by reference to the loss or gain of, optionally engineered, sites recognised by restriction enzymes.

According to another aspect of the present invention there is provided a human cMOAT gene or its complementary strand comprising a variant allelic polymorphism at one or more of positions defined herein or a fragment thereof of at least 20 bases comprising at least one novel polymorphism.

Fragments are at least 17 bases, more preferably at least 20 bases, more preferably at least 30 bases.

According to another aspect of the present invention there is provided a polynucleotide comprising at least 20 bases of the human cMOAT gene and comprising an allelic variant selected from any one of the following:

| | Variant |
|---|---|
| Region | SEQ ID NO: 1 |
| coding | 78 T |
| | 1350 A |
| | 1584 G |
| | 1686 T |
| | 2647 G |
| | 3208 C |
| | 3664 A |
| | 4391 G |

| | Variant |
|---|---|
| Region | SEQ ID NO: 2 |
| 5′UTR or | 1349 A |
| promoter | |
| | 1875 A |
| | 1879 G |

| Region | Variant |
|---|---|
| SEQ ID NO 3 | 12704 T |
| SEQ ID NO 3 | 29446 T |
| SEQ ID NO 4 | 292 C |
| SEQ ID NO 5 | 232 G |
| SEQ ID NO 5 | 457 G |
| SEQ ID NO 6 | 50 A |
| SEQ ID NO 6 | 68 A |

According to another aspect of the present invention there is provided a human cMOAT gene or its complementary strand comprising a polymorphism, preferably corresponding with one or more the positions defined herein or a fragment thereof of at least 20 bases comprising at least one polymorphism.

Fragments are at least 17 bases, more preferably at least 20 bases, more preferably at least 30 bases.

The invention further provides a nucleotide primer which can detect a polymorphism of the invention.

According to another aspect of the present invention there is provided an allele specific primer capable of detecting a cMOAT gene polymorphism, preferably at one or more of the positions as defined herein.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting a cMOAT gene polymorphism, preferably at one or more of the positions defined herein.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided an allele specific primer or an allele specific oligonucleotide probe capable of detecting a cMOAT gene polymorphism at one of the positions defined herein.

According to another aspect of the present invention there is provided a diagnostic kit comprising an allele specific oligonucleotide probe of the invention and/or an allele-specific primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the single nucleotide polymorphisms of this invention may be used as genetic markers in linkage studies. This particularly applies to the polymorphisms at 78 and 1350 in SEQ ID NO 1 because of their relatively high frequencies (see below). The cMOAT gene has been mapped to chromosome 10q24 (Tanaguchi et al (1996). Cancer Res. 56, 4124-4129.).

Low frequency polymorphisms may be particularly useful for haplotyping as described below. A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms which are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine. Ann Hum Genet (1998) 62:481-90, De Stefano V, Dekou V, Nicaud V, Chasse J F, London J, Stansbie D, Humphries S E, and Gudnason V; and Variation at the von willebrand factor (vWF) gene locus is associated with plasma vWF:Ag levels: identification of three novel single nucleotide polymorphisms in the vWF gene promoter. Blood (1999) 93:4277-83, Keightley A M, Lam Y M, Brady J N, Cameron C L, Lillicrap D).

According to another aspect of the present invention there is provided a computer readable medium comprising at least one novel sequence of the invention stored on the medium. The computer readable medium may be used, for example, in homology searching, mapping, haplotyping, genotyping or pharmacogenetic analysis.

According to another aspect of the present invention there is provided a method of treating a human in need of treatment with a drug transportable by cMOAT in which the method comprises:
i) diagnosis of a polymorphism in cMOAT in the human, which diagnosis preferably comprises determining the sequence at one or more of the following positions:
positions 78, 1350, 1584, 1686, 2647, 3208, 3664 and 4391 in the coding region of the cMOAT gene as defined by the position in SEQ ID NO: 1;
positions 1349, 1875 and 1879 in the 5′UTR region of the cMOAT gene as defined by the position in SEQ ID NO: 2;
positions 12704 and 29446 in the intron region of the cMOAT gene as defined by the position in SEQ ID NO: 3;
position 292 in the intron region of the cMOAT gene as defined by the position in SEQ ID NO: 4;
positions 232 and 457 in the intron region of the cMOAT gene as defined by the position in SEQ ID NO: 5;
positions 50 and 68 in the intron region of the cMOAT gene as defined by the position in SEQ ID NO: 6; and
positions 417, 495, 529, 849, 1036 and 1188 in the cMOAT polypeptide as defined by the position in SEQ ID NO: 7.
and determining the status of the human by reference to polymorphism in the cMOAT gene; and
ii) administering an effective amount of the drug.

Preferably determination of the status of the human is clinically useful. Examples of clinical usefulness include deciding which statin drug or drugs to administer and/or in deciding on the effective amount of the statin drug or drugs. Statins already approved for use in humans include atorvastatin, cerivastatin, fluvastatin, pravastatin and simvastatin. The reader is referred to the following references for further information: Drugs and Therapy Perspectives (12^{th} May 1997), 9: 1-6; Chong (1997) Pharmacotherapy 17: 1157-1177; Kellick (1997) Formulary 32: 352; Kathawala (1991) Medicinal Research Reviews, 11: 121-146; Jahng (1995) Drugs of the Future 20: 387-404, and Current Opinion in Lipidology, (1997), 8, 362-368. Another statin drug of note is compound 3a (S4522) in Watanabe (1997) Bioorganic and Medicinal Chemistry 5: 437-444. The term “drug transportable by cMOAT” means that transport by cMOAT in humans is an aspect of a drug exerting its pharmaceutical effect in man. For example, some statins may be transported by cMOAT as conjugates in the human body.

According to another aspect of the present invention there is provided an allelic variant of human cMOAT polypeptide comprising at least one of the following:
a isoleucine at position 417 of SEQ ID NO 7;
a glutamic acid at position 495 of SEQ ID NO 7;
a tryptophan at position 529 of SEQ ID NO 7;
a arginine at position 849 of SEQ ID NO 7;
a threonine at position 1036 of SEQ ID NO 7; and
a valine at position 1188 of SEQ ID NO 7;
or a fragment thereof comprising at least 10 amino acids provided that the fragment comprises at least one allelic variant.

Fragments of polypeptide are at least 10 amino acids, more preferably at least 15 amino acids, more preferably at least 20 amino acids.

According to another aspect of the present invention there is provided an antibody specific for an allelic variant of human cMOAT polypeptide as described herein.

Antibodies can be prepared using any suitable method. For example, purified polypeptide may be utilized to prepare specific antibodies. The term “antibodies” is meant to include polyclonal antibodies, monoclonal antibodies, and the various types of antibody constructs such as for example F(ab′)₂, Fab and single chain Fv. Antibodies are defined to be specifically binding if they bind the allelic variant of cMOAT with a Kₐ of greater than or equal to about 10⁷ M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the invention there is provided a diagnostic kit comprising an antibody of the invention.

The invention will now be illustrated but not limited by reference to the following Examples. All temperatures are in degrees Celsius.

In the Examples below, unless otherwise stated, the following methodology and materials have been applied.

AMPLITAQ™ available from Perkin-Elmer Cetus, is used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989) or in “Current Protocols in Molecular Biology Volumes 1-3, edited by F M Asubel, R Brent and R E Kingston; published by John Wiley, 1998.

Electropherograms were obtained in a standard manner: data was collected by ABI377 data collection software and the wave form generated by ABI Prism sequencing analysis (2.1.2).

### EXAMPLE 1

Identification of Polymorphisms

1. Methods

DNA Preparation

DNA was prepared from frozen blood samples collected in EDTA following protocol I (Molecular Cloning: A Laboratory Manual, p392, Sambrook, Fritsch and Maniatis, 2nd Edition, Cold Spring Harbor Press, 1989) with the following modifications. The thawed blood was diluted in an equal volume of standard saline citrate instead of phosphate buffered saline to remove lysed red blood cells. Samples were extracted with phenol, then phenol/chloroform and then chloroform rather than with three phenol extractions. The DNA was dissolved in deionised water.

Template Preparation

Templates were prepared by PCR using the oligonucleotide set out below. The annealing temperature for all sets of primers was 58° C. followed by an extension temperature was 72° C. and a denaturation temperature of 94° C. Generally 50 ng of genomic DNA was used in each reaction and subjected to 35 cycles of PCR.

All primers used for amplification of the exons are as published by Toh et al. (Am. J. Hum. Genet., 1999, 64:739-746).

Primers for the amplification of the promoter and 5′UTR are listed below: Primer sequences are based on SEQ ID NO 2.

| Fragment | Forward Oligo | Reverse Oligo | Annealing Temp | Time |
|---|---|---|---|---|
| 1164-1729 | 1164-1183 | 1710-1729 | 58° C. | 60 s |
| 1622-2164 | 1622-1641 | 2145-2164 | 58° C. | 60 s |
| 2061-2677 | 2061-2080 | 2658-2677 | 58° C. | 60 s |

Primers for the amplification of the 3′UTR are listed below and are based on SEQ ID NO 1.

| Fragment | Forward Oligo | Reverse Oligo | Annealing Temp | Time |
|---|---|---|---|---|
| 4707-5207 | 4707-4726 | 5188-5207 | 58° C. | 60 s |

For dye-primer sequencing these primers were modified to include M13 forward and reverse primer sequences (ABI protocol P/N 402114, Applied Biosystems) at the 5′ end of the forward and reverse oligonucleotides respectively.

Dye Primer Sequencing

Dye primer sequencing using M13 forward and reverse primers was as described in the ABI protocol P/N 402114 for the ABI Prism™ dye primer cycle sequencing core kit with “AmpliTaq FS” DNA polymerase, modified in that the annealing temperature was 45° C. and DMSO was added to the cycle sequencing mix to a final concentration of 5%.

The extension reactions for each base were pooled, ethanol/sodium acetate precipitated, washed and resuspended in formamide loading buffer.

4.25% Acrylamide gels were run on an automated sequencer (ABI 377, Applied Biosystems).

2. Results

Polymorphisms

| | | Protein | | |
|---|---|---|---|---|
| Position | DNA | polymorphism | Allele | Allele |
| SEQ ID 1 | polymorphism | SEQ ID NO 7 | frequency | frequency |
| 78 | C/T | none | C 72.7% | T 27.3% |
| 1350 | G/A | Val417Ile | GTT 77.5% | ATT 22.5% |
| 1584 | A/G | Lys495Glu | AAA 97.8% | GAA 2.2% |
| 1686 | C/T | Arg529Trp | CGG 97.8% | TGG 2.2% |
| 2647 | T/G | Leu849Arg | CTG 94.4% | CGG 5.6% |
| 3208 | T/C | Ile1036Thr | ATA 93.5% | ACA 6.5% |
| 3664 | T/A | Val1188Glu | GTG 95.5% | GAG 4.5% |
| 4391 | T/G | none | T 4.3% | G 95.7% |

| Position | DNA | Allele | Allele |
|---|---|---|---|
| SEQ ID NO 2 | polymorphism | frequency | frequency |
| 1349 | G/A | G 57.5% | A 42.5% |
| 1875 | G/A | G 80% | A 20% |
| 1879 | A/G | A 52.6% | G 47.4% |

| Position | DNA | | |
|---|---|---|---|
| SEQ ID NO 3 | polymorphism | Allele frequency | Allele frequency |
| 12704 | C/T | C 58.7% | T 41.3% |
| 29446 | T/C | T 95.6% | C 4.4% |

| Position | DNA | Allele | Allele |
|---|---|---|---|
| SEQ ID NO 4 | polymorphism | frequency | frequency |
| 292 | T/C | T 58.9% | C 41.1% |

SEQ ID NO 4 is exon 15 and intronic sequence flanking both ends thereof.

| Position | DNA | Allele | Allele |
|---|---|---|---|
| SEQ ID NO 5 | polymorphism | frequency | frequency |
| 232 | C/G | C 60.9% | G 39.1% |
| 457 | A/G | A 93.8% | G 6.2% |

SEQ ID NO 5 is exon 27/intron 27/exon 28 and intronic sequence flanking both ends thereof.

| Position | DNA | Allele | Allele |
|---|---|---|---|
| SEQ ID NO 6 | polymorphism | frequency | frequency |
| 50 | G/A | G 97.8% | A 2.2% |
| 68 | G/A | G 89.3% | A 10.7% |

SEQ ID NO 6 is exon 30 and intronic sequence flanking both ends thereof.
Allele frequencies were based on analysis of 20 to 23 individuals.

### EXAMPLE 2

Diagnostic Assays for Polymorphisms within the 5′End of the cMOAT Gene.

The cMOAT 5′end is set out in SEQ ID NO 2 and all positions in this Example 2 relate to the position therein. DNA and template preparation as before; see Example 1.

Position 2874 C/T

Diagnostic Primer 2875-2903

The diagnostic primer contains a 2 mismatches from the wild type sequence at positions 25 (G→T) and position 27 (A→T).
Constant Primer 2624-2643

PCR amplification using these primers will generate a product of 280 bp. The use of the diagnostic primer on a template creates a Bsr GI recognition sequence (TGTACA) at the site of the polymorphism. Bsr GI (New England Biolabs) can therefore distinguish the two polymorphic variants. Digestion of PCR products was performed according to the manufacturers instructions (New England Biolabs).

| | | |
|---|---|---|
| ...CTT CGT ACA... | wild type | uncut |
| ...CTT TGT ACA... | variant | cut |

Position 1875 G/A
Diagnostic Primer 1849-1874

The diagnostic primer contains a 2 mismatches from the wild type sequence at positions 22 (C→T) and position 25 (A→T).
Constant Primer 2145-2164

PCR amplification using these primers will generate a product of 315 bp. The use of the diagnostic primer on a template creates a Bsp H1 recognition sequence (TCATGA) at the site of the polymorphism. Bsp H1 (New England Biolabs) can therefore distinguish the two polymorphic variants. Digestion of PCR products was performed according to the manufacturers instructions (New England Biolabs).

| | | |
|---|---|---|
| ...AGG TCA TGG... | wild type | uncut |
| ...AGG TCA TGA... | variant | cut |

Position 1879 A/G
Diagnostic Primer 1880-1903

The diagnostic primer contains a 2 mismatches from the wild type sequence at positions 22 (C→G) and position 24 (T→A).
Constant Primer 1622-1641

PCR amplification using these primers will generate a product of 315 bp. The use of the diagnostic primer on a template creates a NsiI recognition sequence (ATGCAT) at the site of the polymorphism. NsiI (New England Biolabs) can therefore distinguish the two polymorphic variants. Digestion of PCR products was performed according to the manufacturers instructions (New England Biolabs).

| | | |
|---|---|---|
| ...ACA ATC CTT... | wild type | uncut |
| ...ACA ATG CAT... | variant | cut |

### EXAMPLE 3

Diagnostic Assays for Polymorphisms within the Coding Sequence of the cMOAT Gene.

The cMOAT gene sequence is set out in SEQ ID NO 1 and all positions in this Example 3 relate to the position therein unless stated otherwise.

Position 1350 G/A

Diagnostic Primer 1352-1379 (SEQ ID NO: 1)
   The diagnostic primer contains 1 mismatch from the wild type sequence at position 28 (T→C)
Constant Primer 23107-23128 (SEQ ID NO: 3)

PCR amplification using these primers will generate a product of 201 bp. The use of the diagnostic primer on a template creates a Nco I recognition sequence (CCATGG) at the site of the polymorphism. Nco I (New England Biolabs) can therefore distinguish the two polymorphic variants. Digestion of PCR products was performed according to the manufacturers instructions (New England Biolabs).

| | | |
|---|---|---|
| ...ACA CCG TTG... | wild type | uncut |
| ...ACA CCA TTG... | variant | cut |

Position 1686 C/T

Amplification is carried out using primers described below for the amplification of exon 12. PCR amplification with these primers will generate a product of approximately 500 bp. The polymorphism destroys a natural AccIII recognition sequence (TCCGGA) at the site of the polymorphism. AccIII (New England Biolabs) can therefore distinguish the two polymorphic variants. Digestion of PCR products was performed according to the manufacturers instructions (New England Biolabs).

| | | |
|---|---|---|
| ...ACC TCC GGA... | wild type | cut |
| ...ACC TCT GGA... | variant | uncut |

Position 2647 T/G

Amplification is carried out using primers described below for the amplification of exons 18 and 19. PCR amplification with these primers will generate a product of approximately 780 bp. The polymorphism destroys a natural EcoRII recognition sequence (CCWGG where W represents A or T) at the site of the polymorphism. EcoRII (New England Biolabs) can therefore distinguish the two polymorphic variants. Digestion of PCR products was performed according to the manufacturers instructions (New England Biolabs).

| | | |
|---|---|---|
| ...CTC TCC TGG... | wild type | cut |
| ...CTC TCC GGG... | variant | uncut |

Position 3208 T/C

Amplification is carried out using primers described below for the amplification of exons 22 and 23. PCR amplification with these primers will generate a product of approximately 750 bp. The polymorphism creates a natural RsaI recognition sequence (GTAC) at the site of the polymorphism. RsaI (New England Biolabs) can therefore distinguish the two polymorphic variants. Digestion of PCR products was performed according to the manufacturers instructions (New England Biolabs).

| | | |
|---|---|---|
| ...AAT GTA TCT... | wild type | uncut |
| ...AAT GTA CCT... | variant | cut |

Nucleotide 3664 T/A
Diagnostic Primer 3665-3691

The diagnostic primer contains 1 mismatch from the wild type sequence at position 25 (C→G)

Constant Primer within intron 24 was as published in Toh et al. Am. J. Hum. Genet., 1999, 64:739-746.

PCR amplification using these primers will generate a product of 241 bp. The use of the diagnostic primer on a template destroys a Tsp 45I recognition sequence (GTSAC where S is either G or C) at the site of the polymorphism. Tsp 45I (New England Biolabs) can according to the manufacturers instructions (New England Biolabs).

| | | |
|---|---|---|
| ...GAG GTG ACG... | wild type | cut |
| ...GAG GAG ACG... | variant | uncut |

Position 4391 T/G
Diagnostic Primer 4392-4112

The diagnostic primer contains 2 mismatches from the wild type sequence at positions 18 (C→A) and 19 (G→C).
Constant Primer 4252-4272

PCR amplification using these primers will generate a product of 163 bp. The use of the diagnostic primer on a template creates a Sna BI recognition sequence (TACGTA) at the site of the polymorphism. Sna BI (New England Biolabs) can therefore distinguish the two polymorphic variants. Digestion of PCR products was performed according to the manufacturers instructions (New England Biolabs).

| | | |
|---|---|---|
| ...AGT TAC TTA... | wild type | uncut |
| ...AGT TAC GTA... | variant | cut |

cMOAT Exon-specific oligos were from Toh et al. Am. J. Hum. Genet., 1999, 64:739-746
Promoter & 5′UTR Oligonucleotides (SEQ ID NO 2; F=Forward, R=Reverse)
Product 1F (1164-1183)
Product 1R (1710-1729)
Promoter 2F (1622-1641)
Product 2R (2145-2164)
Product 3F (2061-2080)
Product 3R (2658-2677)
   3′UTR Oligonucleotides (SEQ ID NO: 1; F=Forward, R=Reverse)
3′UTR F (4707-4726)
3′UTR R (5188-5207)

1. A method for the diagnosis of a polymorphism in cMOAT in a human, which method comprises determining the sequence of the human at one or more of the following positions:
positions 78, 1350, 1584, 1686, 2647, 3208, 3664 and 4391 in the coding region of the cMOAT gene as defined by the positions in SEQ ID NO: 1;
positions 1349, 1875 and 1879 in the 5′UTR region of the cMOAT gene as defined by the positions in SEQ ID NO:2;
positions 12704 and 29446 in the intron region of the cMOAT gene as defined by the positions in SEQ ID NO:3;
positions 292 in the intron region of the cMOAT gene as defined by the position in SEQ ID NO:4;
positions 232 and 457 in the intron region of the cMOAT gene as defined by the positions in SEQ ID NO:5;
positions 50 and 68 in the intron region of the cMOAT gene as defined by the positions in SEQ ID NO:6; and
positions 417, 495, 529, 849, 1036 and 1188 in the cMOAT polypeptide as defined by the positions in SEQ ID NO:7,
and determining the status of the human by reference to the polymorphism in cMOAT.

2. Use of a diagnostic method as defined in claim 1 to assess the pharmacogenetics of a drug transportable by cMOAT.

3. A polynucleotide comprising at least 20 bases of the human cMOAT gene and comprising an allelic variant selected from any one of the following:

| | Variant |
|---|---|
| Region | SEQ ID NO: 1 |
| Coding | 78 T |
| | 1350 A |
| | 1584 G |
| | 1686 T |
| | 2647 G |
| | 3208 C |
| | 3664 A |
| | 4391 G |
| Region | Variant SEQ ID NO: 2 |
|---|---|
| 5′UTR or promoter | 1349 A |
| | 1875 A |
| | 1879 G |
| Region | Variant |
|---|---|
| SEQ ID NO: 3 | 12704 T |
| SEQ ID NO: 3 | 29446 T |
| SEQ ID NO: 4 | 292 C |
| SEQ ID NO: 5 | 232 G |
| SEQ ID NO: 5 | 457 G |
| SEQ ID NO: 6 | 50 A |
| SEQ ID NO: 6 | 68 A |

4. A nucleotide primer which can detect a polymorphism as defined in claim 1.

5. An allele specific primer capable of detecting a cMOAT gene polymorphism as defined in claim 1.

6. An allele-specific oligonucleotide probe capable of detecting a cMOAT gene polymorphism as defined in claim 1.

7. Use of a cMOAT gene polymorphism as defined in claim 1 as a genetic marker in a linkage study.

8. A method of treating a human in need of treatment with a drug transportable by cMOAT in which the method comprises:
i) diagnosis of a polymorphism in cMOAT in the human, which diagnosis preferably comprises determining the sequence at one or more of the following positions:
positions 78, 1350, 1584, 1686, 2647, 3208, 3664 and 4391 in the coding region of the cMOAT gene as defined by the positions in SEQ ID NO: 1;
positions 1349, 1875 and 1879 in the 5′UTR region of the cMOAT gene as defined by the positions in SEQ ID NO:2;
positions 12704 and 29446 in the intron region of the cMOAT gene as defined by the positions in SEQ ID NO:3;
position 292 in the intron region of the cMOAT gene as defined by the position in SEQ ID NO:4;
positions 232 and 457 in the intron region of the cMOAT gene as defined by the positions in SEQ ID NO:5;
positions 50 and 68 in the intron region of the cMOAT gene as defined by the positions in SEQ ID NO:6; and
positions 417, 495, 529, 849, 1036 and 1188 in the cMOAT polypeptide as defined by the positions in SEQ ID NO:7,
and determining the status of the human by reference to the polymorphism in cMOAT; and
ii) administering an effective amount of the drug.

9. An allelic variant of human cMOAT polypeptide comprising at least one of the following:
an isoleucine at position 417 at SEQ ID NO:7;
a glutamic acid at position 495 of SEQ ID NO:7;
a tryptophan at position 529 of SEQ ID NO:7;
an arginine at position 849 of SEQ ID NO:7;
a threonine at position 1036 of SEQ ID NO:7;
a valine at position 1188 of SEQ ID NO:7;
or a fragment thereof comprising at least 10 amino acids provided that the fragment comprises at least one allelic variant.

10. An antibody specific for an allelic variant of human cMOAT polypeptide as defined in claim 9.


SEQUENCE LISTING (7 sequences)

SEQ ID NO: 1 - DNA, 5300 bp, Homo sapiens
aggataattcctgttccactttctttgatgaaacaagtaaagaagaaacaacacaatcatattaatagaagagtcttcgttccagacgcagtccaggaatcatgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggagaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagttacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcataccatcatggacagtgacaaggtaatggtcctagacaacgggaagattatagagtacggcagccctgaagaactgctacaaatccctggacccttttactttatggctaaggaagctggcattgagaatgtgaacagcacaaaattctagcagaaggccccatgggttagaaaaggactataagaataatttcttatttaattttattttttataaaatacagaatacatacaaaagtgtgtataaaatgtacgttttaaaaaaggataagtgaacacccatgaacctactacccaggttaagaaaataaatgtcaccaggtacttgagaaacccctcgattgtctacctcgatcgtacttccttgctacccacccctcccagggacaaccactgtcctgaatttcacgataattattcctttgcctttcatttctgttttatcacctttgtatgtatctttaaacaacatatacccttttttacttatgtaaatggactgactcatactgcatacatcttctatgacttgattcttttgttcaatattatatctgagattcatccatggtgatgcaaataggtgcattattttttttcactgctctgtagtctggcattgtatgaatacagcacaatgtatcagttttaatattggggatcattagcattattctcaggtttttaaaaattataagcagtactactatgg

SEQ ID NO: 2 - DNA, 3000 bp, Homo sapiens
actcatgaaagtgtggagatcatcccctccttatggctgagtcactctggaatttttctctctcaaacttatctacactgggcctccggcaattcaattacagttcaggttttcctgccctagccctggttcctgctgagatttcttttcttttttcttttttttttttttgagacagtgtctttgttgcccaggctggagtgcagtggcgtgatctcaagtcactgcaagctccgcttcccgggttcacgccattctcctgcctcagcatctccgagtagctgggactacaggcgcccggcaccacgcccggccaattttttgtatttttagtagagacagggtttcactgtggtctcgatctcctgacctcgtgatccgcccgcctcggcctcccaaagtgctgggattacaagcgtgagccaccgcgcccggcccctgctgagatttctgcgccagtaaattgtgatttttgatattctcttgtttatctcttcagtttgccctgtaaccttatctctctacagatctaagaagagttattgatttttctatttgttcagatttttagttattgttaggaaagattcatgacttcctggctccttacttgcttttctcattcaatattccctatgagtacatattttatactttaagcaacttgttttgatgaacatttagattctccaccctctctttttttgattacttagaacgtttaggccagacacgatggctcatgcctgtaatcccagcagtttgggaggccgaggcgaatggatcgcttgagcccaggagtttgaggctctacaaaaaaattaaaaaaagaaaattaggtgttaatccttgaccttatatttctcaaagttttaccaagagttcccccggactccgagtcctaccaccagtgccaagagaagtatgcagcctaaaggtgcaccctgccccaagtgagatctcttagttccacagctgacctttcctggctcttctggtgagtggtatggaaaatgatggagaaactgccatatgttttcataatggtcagaccaatttacatttccatcaacagtatactagggttcccttttctccacacccttgccagcacttatcttgttgtttttttttttttttgtaatagccatcctaataggtatgaggtggtatctaactgtggttttgatttgtgttttccagatgacttgtgaagttgattcagttgtcatatacctgttggccatttatatgtcttctttggaaaagtgtctgttcaagtcctttgcccactttttaatttgttagtgtatgtttgctattgagttgtatgagttccttatagtatgttgtggatattaactcttcatcagttatatggtctgcaaatattttctcccacactgaatgctgccttttcattttttttatgtgtatacttgacttttaaaatcagtaacaaggtccatacaattcacatttggctgatatgcctcttgattctcttgtaatttgtaagttcctttgactcctttccttcttttaatttatttattaaaaaatggaatcatttgcactatagaatctcccacattctggattttgacaattgcattccaatggagtcctttagcatgttcctgtgtttcttataatccggtagttagatctagaatttgaccagatttaaggccaattttttttagctaggatactgcatgggtggttatgttttagctaggataccgcatgggtggttcatgatatcatgaaaaaagcatgttcttaaagcaatttaagtgacagtacaaaaggttgggtcaggtggggcacggtagctcatgcctgcaatcccagccctttgggaggccaaggcagaaggattgttgaagcctggagtttgagaccagcctgggcaacatagtgagaccccgtccctacagaaaacatttttttaattagctgggcttgttggcatgtgcctgtagtcccagctactcaggaagctgaggcaggaagatcgcttgaacccatgaggtcaaggctgcaatgaatcatgatggcaacactgcactctagctcaggcaacagaccaagaccctgtctcaaaaaacaaaaaacaataacaaaaaaagaaaaggttgggtcgatgacagtttctagcgactgatgccaccactctgtttacagaatcacccctctcctctggccctgatgttttatcatgatatcatatttacaatgcctggcaaaggcttcattcccatttggcatactacctctcaggcaaatagaacttttgaaagcctgtattatgtatataacatataggctcacactggataagctattttataacctgacttcttcaaagaaagtttacatcatgtttaaaccatgttttagattctatatttttaattaaaaatctaaggaagaaggatatttcacatttctataaactctaagatcttgcagcagaagcgaaactgcacatttaggggtgcctgcccctctactgatgctgccctttgtgggtcatatgtccttaggaaaatgaaagactgtgcactcttgatttgttggccagctctgttgacatctttcagtggttccttttatgtatggccactcctacagaggcctcttgtactttgggaactggtgagtctccctgtccctagggctttttagtcacatgtccatccactgtttcaatgtaacatgcatctaggcaaggttaacgattaaatggttgggatgaaaggtcatcctttacggagaacatcagaatggtagataattcctgttccactttctttgatgaaacaagtaaagaagaaacaacacaatcatattaatagaagagtcttcgttccagacgcagtccaggaatcatgctggagaagttctgcaactctactttttgggtgagaaattacatttatcttcatattgactcttctcagactcagaacaagtggtagttagttaacttag

SEQ ID NO: 3 - DNA, 29844 bp, Homo sapiens
accttatatttctcaaagttttaccaagagttcccccggactccgagtcctaccaccagtgccaagagaagtatgcagcctaaaggtgcaccctgccccaagtgagatctcttagttccacagctgacctttcctggctcttctggtgagtggtatggaaaatgatggagaaactgccatatgttttcataatggtcagaccaatttacatttccatcaacagtatactagggttcccttttctccacacccttgccagcacttatcttgttgttttttttttttttttgtaatagccatcctaataggtatgaggtggtatctaactgtggttttgatttgtgttttccagatgacttgtgaagttgattcagttgtcatatacctgttggccatttatatgtcttctttggaaaagtgtctgttcaagtcctttgcccactttttaatttgttagtgtatgtttgctattgagttgtatgagttccttatagtatattgtggatattaactcttcatcagttatatggtctgcaaatattttctcccacactgaatgctgccttttcattttttttatgtgtatacttgacttttaaaatcagtaacaaggtccatacaattcacatttggctgatatgcctcttgattctcttgtaatttgtaagttcctttgactcctttccttcttttaatttatttattaaaaaatggaatcatttgcactatagaatctcccacattctggattttgacaattgcattccaatggagtcctttagcatgttcctgtgtttcttataatccggtagttagatctagaatttgaccagatttaaggccaattttttttagctaggatactgcatgggtggttatgttttagctaggataccgcatgggtggttcatgatatcatgaaaaaagcatgttcttaaagcaatttaagtgacagtacaaaaggttgggtcaggtggggcacggtagctcatgcctgcaatcccagccctttgggaggccaaggcagaaggattgttgaagcctggagtttgagaccagcctgggcaacatagtgagaccccgtccctacagaaaacatttttttaattagctgggcttgttggcatgtgcctgtagtcccagctactcaggaagctgaggcaggaagatcgcttgaacccatgaggtcaaggctgcaatgaatcatgatggcaacactgcactctagctcaggcaacagaccaagaccctgtctcaaaaaacaaaaaacaataacaaaaaaagaaaaggttgggtcgatgacagtttctagcgactgatgccaccactctgtttacagaatcacccctctcctctggccctgatgttttatcatgatatcatatttacaatgcctggcaaaggcttcattcccatttggcatactacctctcaggcaaatagaacttttgaaagcctgtattatgtatataacatataggctcacactggataagctattttataacctgacttcttcaaagaaagtttacatcatgtttaaaccatgttttagattctatatttttaattaaaaatctaaggaagaaggatatttcacatttctataaactctaagatcttgcagcagaagcgaaactgcacatttaggggtgcctgcccctctactgatgctgccctttgtgggtcatatgtccttaggaaaatgaaagactgtgcactcttgatttgttggccagctctgttgacatctttcagtggttccttttatgtatggccactcctacagaggcctcttgtactttgggaactggtgagtctccctgtccctagggctttttagtcacatgtccatccactgtttcaatgtaacatgcatctaggcaaggttaacgattaaatggttgggatgaaaggtcatcctttacggagaacatcagaatggtagataattcctgttccactttctttgatgaaacaagtaaagaagaaacaacacaatcatattaatagaagagtcttcgttccagacgcagtccaggaatcatgctggagaagttctgcaactctactttttgggtgagaaattacatttatcttcatattgactcttctcagactcagaacaagtggtagttagttaacttagggtggtcaccaacaagaaccagaaatgttagatgtgcaattggtctaaagctcaggctttttagaaagcataattcataatatctccagaggatataagatctgggaaatgtaatgcaaattattctctgtcatatgtttttttatatcagactcttgtactagctagtcgcaaaattgccttctctaaagagaaacaggtaaaaaccttccaatttaatccgtgtgaggaaggagaattgttgttgcatactttgctagtagaaggaagagaagatccgtgactgagtcttgctgagacatgttgatgttgaaatgataatgataagttctagaactggcaactaatgatatggactagaagttgagtctagggatgcaactttgggcatcatgagcattgaaataatttttgaacaactagagttgggtaatattgtttttaaggaaaagaagagacaccacttagagggtgaacaagaacagtgtaacattacggagtcatagatagcaaaaaatttcaagataggggccaatcaacagtatcaaaggtctagtggaatagaaaacaaaaaaggaccttagatttagtgattttagtaagggtttggtgagaaagtatcatctgcaaactcctagcctcagcatcagaatcacctggggcaataaatgtagtttaagtatcttaagtgattcttctgaattctaacatttgataaccttatatagaggtgagaggggcagggatgagatagcagagcacctgggcagcacctcgtgtcttagaaaggacctgggtcccaagaccctcgaggccaggagtgtataccacggcaaggtggatcttcatgggctatagtatctagacaggccatctcttgagaattctttctgagaactacctgacaaactttcttggtcagggcatcgcaagcaattctttaaagagtctgtataaataatcatataaaacaatttttaactatttataggaaaagaaaaatccacactcatgtgactcagatacaagaattggtgcccaccccccaccccccaacttccacttcacaagacacttccatttatcgcagaatgcaaagactgcacaaaacttgctggttggaagccttctgtccaacaatgctgttgtcactgctgtgctgaaactggccaggtttttaatgtaagcgctttgggccatcagagagcaaatgttacagtatgactaagcattctccctgcatggcgcctgcatgtttgaatatctgccagtgagttcagttagtgctagaaacttgaataattttttatatcccagggaactattctgtcctttaaactggggaacagtccttcttcatcctataatttggcaaatgcagtggtgaggtttactcagaatagctggtacaaaattcacttagaatcacaagaagacacgtttttggagggtgggttggccagaaagtgactacatttgtcattaaatggtgaatttgtatccatgtgcacaaataggaaaatacggatagttaaagggtaaggctggatatggatatggagttgaatttaaattgtgtgaaagcagtgggatgtgctgcagggtggttttctgtcttcacaatgcatgtatgcaacaatccttcccctttggtctccagaattcctcattcctggacagtccggaggcagacctgccactttgttttgagcaaactgttctggtgtggattcccttgggctacctatggctcctggccccctggcagcttctccacgtgtataaatccaggaccaagagatcctctaccaccaaactctatcttgctaagcaggtaaagttaacaccactgtttctgaactctaattccttggtgcacagtagagacattttcttggtggttagtgttctcttccttgtctttaagcagctgtcccaggtgccattgtctcaggtagagccaggctcaaggtttccctcacgggtcccttgctgcatttcttaaatcaaagtggctttgatttttgcataagaatggtgactcttaattcttgtggatattgggttccctaaacacaaagggcatataatcaatcaaattatctttatagcatttgagaaatgaacactggatcacctgtctttaaacagctctaataaatcagtggactgtaattcagttggcatctacctcgtgaaaatgagctagataatggatctcctgccccactgaggcaatccaactatcactcatcttcaaaaacttgggtccgaaataaggtttccattttccccgcatatttgatgagtcaggatgatcactcacagtagaaggaggaggggtaagtgtgcacagtgacaagcacgagtagggaaataatggaaaggacaggtctgcgggagaggcccaggaggggccaggaggggaggagccctcagccagctgcaccaactcactcagggaagggtcttgtgggaacccaagaccctcattcgaggttgtaaaattatctcataaaatcttccatctgcttcttttcactatttttttttctatctctccttattatcattattactaccattttaaatggagtctcactctgttgcccaggctggagtgcagtggcactatctcggctcactgcaacctccacctcctgggttcaagcaattcttatgcctcaacccctcccgagtagctgggattacaggagcacgccaccatgtccagctaatttttgtatttttagtagggcgggatttcgccatgttggccagactggtctcgaactcctgacctcaagtgatccctcctgccttggcctcccaaagtgctgcgattacaagcctgagccacctgcacagcctctgtcagtccttatctttcaacaggtttttagaaaggcaaactagattccattgttctactagcatacctaacaatgactcaggttacctggaatcttaaaggaggaaagagcttggccaaaggaagtggatttcaccttgacactttgaggttggggttgatcgtgggttttattagattctgattcaaaaaagaacagtctcagagcttgggacagtgtcactgtctcccttgttgacctgagtgcccatcacaggacctagcatgtttcaaacactcccactggaatacaacatttgttgaatgttggtgttaaatgcattctgggaatagtggttgattcaagagtaggttagagctgacctcagtggtacatacctgtaatcccagttcctctggaagctagaagtggtaggatcactagaggccagcctggcaacatagtgagtccccatctcttaaaaaaataaaataaaataaaaataaaaaggaaagaaacatatgttagactttgaacattctgaacattaaacaactctgaacatttaataacattctcagtacccttggaattgagtcattgttcgaggttagaagcaatcaagttgggaggcttcttctactttctggagcttccaagaagaagctggttaatgagcaaaccaatatcccatcctaataaaagctattagttaactcttttacaaaatactttattgagatataatgtacataccatacagttctctcatttaatacaattcaggccggatgctgtggctcacgcctgtaatcccaacactttgggaggccgaggtgggtggatcacttgaggtcaagagtttgagaccagcttggccaacacagtgaaaccccatctctactaaaaatacaaaaacattcggctgggcatggtggctcacgcttgtaatcccagcactttgggaggccgaggtgggtggatcacgaggtcaggagttcgagacgagcctggccaacatggtgaaaccccgtctctactaaaaatacaaaaattagccaggcatggtggcggacgcctgtaatcccagctactcaggagactgaggcagagaattgcttgaacctgggaggtggaggttgcagtgagctgagatcatgccactgcactccagcctgggtgacagagcaagactccgtctcaaaaaaaaaaaaaaaaattagcctggtgcagtggcacacacctgtaaccccagctactcaggtggctgaggcataaggatcacttgaacccgggaggtggaggttacagtgagccaagatcgtgccactgcactccagcttcagactctgtctcaaaaaaaaaaaaaatgcaattcagtttttttcagtatattaacacatatgtgcaaccatcaccacagtcaattttagaacatttcattatctttaaccccgtctcctccttccccttatccttagcaaccgctaaatctcttttctgtctttatagatttccctgttctagacatttcatatgaatggaattatataatatggttcttttttgattggcttctttcacttagcacaatgtgttcaaggtttatctatgctgtaccattgctataccatgtattgatattttacccctttttttggggggacggagtctcgttctgtcgcccaggcaggctgccagcatagattcctgccaacagtgtgttaaagtgcatgtccttgcaccattgtcagcattgagttattttcaacttttatgtgtaaacatgacatctcactgagttgctttgcatttttatgaatactaacgcaattgtttttttaatttgttaagtgtatttcatttgcgaattgtctgttcatatcacttccttattttttaatgggggtgtgagtgtatttaacttatcttttgaacggataattctcagaggatcacaaagttcaaaaagtacaaaaggagctgggcatagtagctcatgcctgtaatcccaacactttgggaagctgaagtgggagaattgcttgagcccagcatttcgagaccagcctggccaacttggtgagaccgatctctacaaaaaattaaaaaaattcaccaggcatggtggcatgtgcctgtggtcccagctactcgggaggctgaggtgggaagatcacttgacaagttcaaggaggtcaaggctgcagtgagccatgttcacaccactgcacttcagcctgggcgacagaggtagaccctgtctcaaaaaaaaaaagaaaaaaaaggacaaaagggtacccagtactctagtcactcattcatgtcttagttggcaaccattgataccagttcctcatatctccttccagaaatagcctgtgcttttacaaacagctacgtacatatattttttcacacaaatgctagcgtcccatatgtgcttttctgtgtcttagagctccttccattttagcacataaaaatctgcatttttctctctttttgtggatattttattcatttgtacctaagcagccaaactcgtactgataccaccatgcagccaaggttactgaaacctctttgattagacttcagtcatatcccacccccaaatagacccaataggtacaatgaaaaccagtatatcaaggcagaatacagagctgtaaaacaagaaaaatcctatgagggagggagggaaaaaggaagaattccgccactacccagactaaaatcgagttaatatattcatctccacacgttttcaggctgcatgttcatggagtaatttaggaataaacctatgatttgtgaagcactgaaatacttagtggtttgctgtattacatcatagctttccccagcagtttccaagacagcttccaaaaactggcaggagaatttcgctggagctgcatgcaggactaccaagattcctccccacagttatgaaaccgatttttccgggactggttgttctagtcttctgggtgccaagtctgacttttctgggaatggttgatccacactggaaggtttccagatgtgttccccaggcattcctggtggtaggaagagtagaggcctgggtattctgtagatgctgagccctgagtcatattgttgcttattctttggtcagttggctttctaccacctcttagctttcattctctgtaactagcttgtatgctgtttgtagctaagattcagggtattggaaagttcttgcagctgctggaggctgcggtgtttctgtctctgaaatccatcattaattacacacagctgggccggagagaacatgattctggtcttctgtttcttctcctggatcttatcttcattcttcactgtgctcttctctgcagaaatgtgtagttttactctgggactagtggaagaattagagctttcctgaataaatagatccacggaggaaggaagatgagagagcctctgagtgggacacctcctcatatgtcatttgcaaggatggataactttcatcagggccacgagtcacaggcattggtgaaagagtccctagaattggatgctttagaataaggcaggctttggagacaagctggactcacactcatgttattgaaaaggagcggggaggggcgggtgcagtggctcacgtctgtaatcccagcactttgggaggctgaggtgggtggatcacctgaggtcagaagttcaagatcagtctgggcaacatggtgaaaccccatctctactaaaaatacaaaaattagccgggcataatggttcacacctgtaatcccatctccttgggaggctgaggcaggagaatcacttgaacctgggaggcggaggttgcagtgagatagcaccactacactccagcctgggtgacagagcgaaactccgtctcaaaaaaaaaaaaaaaaagaaaaaggaaaagaaaaagaaagaaaaggaaaggtggaggaaaattaaagagctggactgtaaaaatggctaaggctttaagatttaagtatctaggtagaatatcttagagaaatatgtaaaatttctccaaatgtaaaagtatcaaaaagccaggatgaagtcagtcccctccacattactgggagacaaccagctcaatcccacaaaaggtctgcttctttctttgtaatgattccttgagatgctgttatttgaatgtaccataatttatgtgtcctgtatgacatgaatttcattggtttctaatattttgcatcatgagcaatgctgccatacacagctttgtatatacttgtgtatacaagtgtgtctataggataaattcccaaaaatgaaatgactgagttaaggatatgtgaactcagaatttttatacacagtgcaaactgccctcaaagaggctttccttggtctcatcaactttgtatcacattttttgatgtttgctaattggttaggcaaaaatgttatgtttcaaattatatatactttcaaattgtattctcatattaatgaggttaagcacatgttcatatgtttaaaagccttttgtgtttttttaaatccatttaatttatatactcatttttttacattgagttattagggtatttttaaaaattactttgtagaagttctttataaagttaactggtcttttttgatatgaactgctcagtgtatttcaaatccattatatgagtttaatatatataatcttatggcaagaccccctccttttccctggcctggggaataggctgctggttagtaccctctagtcatctccaagggcaggtgccttacctctagcttgctctattttacctcctaatttacctaaaggcaaccatagcttaccgcctatgaagctacatttttttcccaaaacctcatagtctcttgtgaggaaaacaccctatagtgtattatatacttgaaaaaatgcatttcagttattaacatgtctttgactctactccttactaaataatagcactgatcctattagttaaagtaaaggtgcattcatctcctttcttggctggttttcattacaaagggcccattatctctgcctgtgccattctggataaattagacttaaaagaatgaataagggtagatggataggggcataaaagcatgaggggaggggagatggtgccacataaatatcttcattttgatggctaagatgatggtatactcagtggaggcagtaattttccccctgataggtggggatgtgggcctttaaaacagtgtaatgtgtatcatgtcttctgagctcataggacaaatactgattggcattgtctgggatcaagactggggctgagtgtggtggcttatgcttgtaatcgcagcagtttgggaggccgaggcaggcagatcacctgagatcaggagcttgagaccagcctggccaacatggtaaaatcctgtctctactaaaaatacaaaaattagccaggtgtggtggcgcacacttgtaatcccagctactcgggaggctgaggcaggagaatcacttgaacccagaaggtggaggttgccgtgagccgagactgcgcctctgcactccagcctgggcaacagagtgagactccgtctcaaaacaaaaacaaaaaacctatggcctaagaaagggaatagtgccttttaaacctagtactttctgttgctatataacaaaataccccaaaacttagcagctaaaagagaattctttaaatcttaacagtttctgtggatcaagaatttgggagcagcttacccaggttctgcttcaaggtctctcaggaggttgaagttcatatgagctgtctggggttactggcatgtgaagtcttgactggggctaaaagatctgcttctaagacgactgtcacatggctggtggcaggaagcctcagtttctcactggctgttggcggaaggtctcagttccttaccaagtggacttctccaaagagctattggagtgtcctcatgactggactggtagctttctccagagtgagcaatcggagacagagaacaaggagaaataatccacattgcttttttatgccctagccttggaagtcacacgctgtcacttctgccattttctattcaccagaagtacatccctaagtccccagcccacactcaagggagagggaattaagctctgctctttgaagagaggaatatcaaaaaatctgcagacatattttaaaacttcacactcagcattcctcaaaaacaatgatcatggcatactttgaacattattctatcacttgaactgtatgtatccattctttccagaaatatttattgtgttatctgaatcactgcataccgcttttcctatccatcaccggaaaccattctgttctaagagccttataaagaatgatatcctcttaacagtggtctttttcccttctcaggtattcgttggttttcttcttattctagcagccatagagctggcccttgtactcacagaagactctggacaagccacagtccctgctgttcgatataccaatccaagcctctacctaggcacatgggtaagacctataccacttctgccctgtttaccttttcattacccataggcatctaggtgaaatgtactctttggggatgaaattaattccaaggtcatctgtctttttcgcgggatccatagtgaggcaaagcttggttggaatggggtgaaacaaattttagcttttcatcttcatttgagcattaatgtatagaggcttcttcctaggttaagtattagctttcatgactaagatgtatatcttatgtccgcacatcttgccatctaagggtcacagaagagagctaagtttcttgatgtaaaacaatattttaaatctgtaaatattgtttttaaattgattgatgactttaaacaatggatcagtgagcttagagtttatataaacttgatatcaaaaaactaatgtctctcttgcaggcattttttcagggggtggggcctagacagtgtttaaatagcaattgaattaattgccatcaattcagtggcaactaatgacacataattatttgaactttcaaactttattgaaaaatcaaaatgtctggcaatcgcaaacctacgttcaggcagagcaatgtcagctgggcctcagtagcagtggctcctttagaccatgtgctcggcacaatccttcctgggcaaatgttataaatcatggctctttccccaccagaatgctgttttaggagcacagctaggtctaggaaatcaacatttaatttattatatctaatgtgagatggtataaggcaagattttttaaaaaaattaattgcttaatcttatattagaaaatcaagctacagacaccttgcacctaattaattagaacctgccttccgaccttactactgcaatgtctgaatttaattttcatgggtcctggaaaggtttttctagctctcaaagtaactccactaaaatgcagatagtgaccttctacattcttggctaacatcccactcaagtgtggtccatggagatgaggcactaaaagaaaaagctggatggcatgggggtgacatgttcaacctggcatctgtgtgctctctacctggccagattagtcacgacagtctcctccctcagccctcctttcttcccatgttctctgacatccttctcccctcagtcctcggttagtggcagtattcttcagaacatcatgtgaatttctctccagctcctggttttgctgatccaatacagcagacaatggtgtgtacagaaaaactcctggttcctgtccctattctggattctctcgatactctgtggcactttccaatttcagactctgatccggacactcttacaggtaaggaaaaaaagagtggatgacatgaggaggtaccatggggcaacctctaactcatagtaaatggcatcaagttgagctccaggatcgaattgtattagagggatttgatcataggctttaatcacaagcattgatatatacgggccatgtagacttcctttggacaaaaggctcatttttcctctttgtttttttcctcatagggtgacaattctaatctagcctactcctgcctgttcttcatctcctacggattccagatcctgatcctgatcttttcagcattttcagaaaataatgagtcatcaaatgtgagattctaaatatgcccatctcatatattacttaattggatgatatcttaatgaatataactttaagatgtctgggtctcacggaggcgccacaagcccagaataaggtagtacagaccattttatgacaaggaaacagatagtgatgagagtaaaatactgacttttactttcacctaccaattgtcagtgtaattaattcattcatatatctatgccagaaaatctgattatcattctactagtggctttagggtctccaaataaacacatgttgataagctgactttaacatcatatctagtttctagcacaacattatatcattaaaaaatcagtttctgattttagagtcccatgaagttcctgtctccaattggtttacatttcgatttttttgtgtctttcagaatccatcatccatagcttcattcctgagtagcattacctacagctggtatgacaggtaggaaagcctggagtatggattggctgtatccttactctctcactcctctgaaagtgtcagaaagattttttattttattattttttttttgagatggagttttgctcttgttgcccaggctggagtgcaatggtgtgatcttggctcactgcaacctccacctcctgggttcaagtgattatcctgcctcagcctcgcaagtagctgggattacaggtgtgagctaccatgcccagctaatttttgtatttttagtagagacggggtttcaccatgttggccaagctggtcttgaactcaagctcatgttccacctgccttggcctctctacgtgctgggattacaggcatgagctacagcgctcagccgaaagattaattttttaaaaatgcctcctgtgacctccatttctcttctacttagagtcagagagtgttaaaacttacagaccaacttaatccaagccctccattgggcagattgggatgctgaggagaagatggagggggatttgtttttggtagcctaggggtcccacaggagaaaagaaaggaggcagtaggacaggaaaggaaaaatttggaggcaggtttggagggtaaggggtgaaatagctggcttgggctaccagaggatctggagcaggcacccttggcctttatagttgcagggacccatttggcaggctggtgaagtctatgaatcccttctcaggaagtgttttaaatgcatgaaataaaatgaaagacattggattctaaagaaaaccaaatatattgaaatatagttaccaaaatattttaaaaacaaatgtgtgatttagtaatacaagtacttctttattaatgcattaaataacaagatctagtgatgagtctaataattaccataattttaaagtagttatgagcataagtgagctatgagaacatgtgggatttctcagcctggcatggtagctcatgcctgtaatcccagcactttgggaggctgaggcgggtggatcacctgaggtcaggagtttgagaccagcctggccaacatggtgaaaccctgtctccactaaaaatacaaaaattagcctggcatggtggcatgtgcctgtaatcccagctccttgggaggctgagacagaagaattgcttgaacctgggaggcagaggttgctgtgagccaagatcgcaccattgcactccagtctgggcgacaagagcaaaattccatcaaaagaaagagagagagagagagaagaaagaaagaaagaaggaaagaaagaaagaaagaaagaaagaaagaaagaaagaaagaaagaaagaaagaaagaaagaaagatttctaaatgctagatttcagttagatgctattacaaataacaatataatttaggccaggtatggtggtttacaactgtaatcctagcactttgggaagccaaggcaggaagatcacttgagcccagaagtttgaggctgcagtcagctatggctctgtcactgaactccagcctgggcagagaaagaccatgtctctaaacatatatataatttattttccatccatattctcaaactcctggctaagagccctgatcaagagagatggcacagtggctggacatagtggctcacacctgtaatcccagcaccttgggaggctgagatgtgcagatcacttgaggtcaggagttggagaacagcctggctaacacggtgaaaccccatctctactaaaaatacaaaaattagctgggggcctggtgcagtggttcacgcctgtaatctcagcactttgggaggcagaggcaggtggatcacctgaggtcaggaatttgagaccagcctggccaacatggtgaaaccctgtctctactaaaaatacaaaattagccaggcgtggtggtgtgtgcctgtaattccagctacttgggaggctgaggcaggagaatcgcttgaacctgggaggcagatcttgcagtaagccgaggtcgcgccactgcactccagcctatcgacaagatcgcaactccgtctcaacagaaacaaaaaaagaagaagaagaacaagaagccgtgtgcagtggtgcaaacctgtaatcccagctacttgggaagcttaagcaggagaatcactttgaagccgggagatggaggttgcggtgagccaagattgcgctactgcactccagcctgggcaacagagtaagactctgtctcaaaaaaaacacaaaaaacaaacaacaacaacaacaaaaaaccacacacaccaaaaacaaagatgccacaggggctgctttatttcttgagggcatcaaggaggcccccaaggagatgccagagtggcagagaggcagccagtgtttgaggaggaaggagcaagagcagggtaaggtgatgctcttttagctgtcttccccttctcttgtcttctaacacattcttttgcccccacccattccttttgctgctctccccactcctctgtcaagttcaaagggccaggagatggggatagtcccattcttctgtccctctatcccagaacctggaggtaggttctgatagaagtggtggagatagcctctgacccagcctgggggtctcagcctgtggttcgctcttgttccagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggtaactttcccttgagtgtctgtgtgagcgcgctgcatgtttcaggcaagggtacatcagcatcatggcgattctgtccttacatttttatagcacttcatacttctcagtgcacttcatattattaatggtattcacaatactgatactaccttagtgatggatacatggcgtgtaatgtactgttctctttcctttggggtacttttgaaattttccagaatgaaacgtggttttgaaaaaaataataccttagcatttctagattttgcaccttggtacaaattagcataagcaggcaatttgttgattcattcacttgaaaatatttattgagcacaactgtgtgtcagtgaccattctgggcactaaaagattgctcatcttttgtgagctagaaaatatgtagtaagcccatacataggttactctttgaaagtaataatagctattaagaaaataaaacaatagtgtatttggagtgaagtgcgacttgagacagggtaacaggggaagcttctcagaggagatgacatctgaacagacactcaaatgatgagatcaggccctctgagggacagtcagagggaagtgtgttccagtcagaaggagcaggcatgcgaggctgagaggggaacacatttggcttgctcagggaatggcaagaagatccaggacagggtgcgcatggggtgagatgggctcaaggaagtggacagggattaagctatgtggacccctgtgggccacaataaggaatctggactttgtctgaagtctgattgccagcggaagattttgagcagaggagtaatgagactggattcatgctgtgaaaagcttacttgggcggctccccagaggatggatcgttgtgggcaagaatggaagctagaagggaggccagggcagttgtcctggcaagggagacccatcagatgggacctcagtgaaagacattgtgagagagtatcactggtaggctgagaagctggaatttggggcaagatgagactcaggaggacataatgaggaggccaaagcatagacagaggctggtgactgagtcgtaatgccccatgtattagtttcctagggctgccatgatagagtaccacaactggatagcttaaaacagaaatgtactgtccccagctcaggaggccagaaggctgaagtcaagatatcagactcgctcccgctgaaacctggaggggaaaatcctctcttccaccttctagtgttccccagcaatgcttggcattccttaacttctggatgcatcactctgacctctgcctccaccatcacaggaccatcttctctctgtgtctgtctcttctcttcttgtaaagatatcaatcatactgcattagggccccagtgacctcatcttaatttggttacatctgcaaagaccttctttcgaaataaggcacattttaaggtattgggggttagggcttcaacatagcttttgggggtggggatacaattcaacccataacatctcacagcaaccatgaaaagaacaagcatgaaaagaacagaaaggacctctcagcatcatgcccaggcccttcactctcaggactgcccctcaggcccccacgcagatgggtctctctttttacatgggactcttaccagcttagttgcctggttttctaatctaaaatagattacaagtatcagttaggagcgggagagtggaacagtgaaggcagctagaagggcagaagcaactaggccagggagagatgatcaaaacctgtacagagaaggccacggggctcacaggctgaccaccctggagctgctcaggccagtaacagacataactctgtggacactgttgtttggttttgtacctaggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaagtgagtctccaggcctcagatggtcctttcagggcctcctctgccaccctcctttttgccactgtgtatgcaagcaggagcttttcttaaattcccattttttaaaaatgctcgtaattttcttccaccttaaccttacagcattgtgttcatgaagaccgcaggcagttttcaagatgtatgatgatatgtactttagcctctctcttttccatgtctgtctcctcatagggggccgggtaattgttaggggctgtttcttgttaaactgctaagggctgagccagatccaagtggcgggtagcatacccctggagagtaactgacttcatctctccatcgcagccttccacgggaggttcaggttaatcttgacaagacttcacaatctgtcatgacctgtggagtgaaccacctgcagggaaagacagggccaaagcattgcagtgacagctaggtggagcatttctgtcattatccattctcacagccatgggttatcaaccctaagctcttgctgctcatctccaagtaaaagccagaacgccaggctggacatggtggctcatgactgtaatctcagcagtttgggagattgaggcaggaagattgtttcaggccaggagttagaaaccaggctggtcaacataacgagaccccatctctacaaaagaaaatagttagctagatgcgatggcgtatgcctgtagacccagctatttaggaggctgaggtgggaggatcacctaagcccaggagttcgaggcttaatgaactatgatcctgccactgcactccagcctgggcaacagagtgagaccctgtatcaaaaacaaaacaaaacagaaaggacactggtaaaactgtagacttaggtctttcaaaggctttggacaattctggtcacttttgttacctacagtgtaaagatagtgtagtctagctggctgtgcatgaggagagaggcatccttggaggccttatgggtataactaacttggcttttctctggcagattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggtaagcagaatacggcaggtatcaccaaagaaaatcccctcagtaaatatgagcattgattccaaggttgaggaaaatgtgatggaaatggtaacttatctcaacacttaatgttgactggccatacagcctcctttagatgtttctttggaaattagattttgagaagtgtgttcacagacaatgggtggagaggccaggagggaaaacgtactccccagggagaagcctgcaagctgttctggtgctcaggatccaccttgcagccctgaaggcttggctacccatccatctcttcctgcctctcttgacaaggtgaaatgccttggaaaagaatataaggtcagaaaagaagctggtgaagggtcaaactcttaaaaccaccaacatttaactcaaatacttaatgaaatccagctcagagacctggtgtatccatctcaaattgcagtcttgccggagctccacctccccagaccaaatttcacctagcaacttctttttctaacctttagtgctaacctaaacctcctctgtctgacttcttttttcagtttgtaaatacattcatgctgattactgagttctgcagacctggaagctttttcatttccttaagatgattttgctcatcttccctctgatacattgtttgttgttgttgttgttttgttttgttttggttgtttttgagatggagtctcgctctatcaccgaggctggagtgcagtggtgcaatcttggctcactgcaacctccatctcctgggttcaagcggttctcctgcctcagcctcccaagcagctgggactacaggcacgtgccaccacgcccggctaattttttgtatttttagtagagatggggtttcaccatgttagccaggatggtctcgatctcctgacgtcatgatctgcccacctcagcctcccaaagtgctgggattataggcatgagccaacgcgcctggccgatacattgtttttaatggttaactttctcaaggaaactgtatgttcctcatgtcctccataccaagatgaagtcccattttgttccttaagaaatgaaaatacagtagttatgatcagtgactacaggcttgaggagaaaattcatccaggaggagataggttccaatctttttataagttggaacactagttgactttcttcctttttttcttccatcactggaaagttccatcatagtaacttctctattacagttcctacttttctcctttgctttcctctctagaaacaccactgtgtctttgtcttccctgagtagatgttggttcgttgagagacagtggcagataatccaagtgtctttaaatcagaagtctagtcaagtcctttgaactcccagataccccccaactggaatttgccttcctccagttgatttttaaaatctccttatttcttctggagatactgagtttctgctggtttacaagttatgaggtcactaaccagcctgtctcagccattgctagtaaagtacacttctacaattttttcataattgtgattattcattcgcactattttggttcttaatattccaggttttgcaactattacaattaataaagagatttataaaatagaaccctcaatgacacttttattcaaataaatatcgctattagaagagtggaagaaaattactaatttatatgtttagacaatgcttcactacttatgaaacattttttacattcattatcaattcacagggctattgccctgtgaaataagtacattatacctattttacagagaaaaatcaggcttagataatttcaatgcctgcaaacacatagccaggagttgtccccagacaagggctactgggactgtatttattgaaaatagaaatatatagtgattaccttgaagggcaattagatgttgttagttgttgtttttttttttttcccctcttccataccaaagggaaggcatagcagtgggttgattcctctagaaccttggtggtctaacattttagaaaactttcttctttttactgtacagccaagaatcctaatatgttccaaagaatgtattggtggattaagatgacagtaatgcttctaggaggagatggaagtagtatgggccacttgtagcctgtacactgtctttgtgggaattacaaggtggctctccttcttctggaaagatgcagtctactccagatggtgccttggtggcatagtgcaagttggtttcagtgctgtggtacatttgtgcaaggcattgttgtaagtgacagacctgtggctatcagcctcctgacctctctaacatgagaacaggaagcccatactacaagatcaggcatctagatagtgcctgaaaaatcctgacacgaaagtcttttattttattttatttatttatttttcaagacggagtctcactctgtagcccaggctggagtgtagtggcatgatctcggctcactgcaacctccgccttccaggttctagcagttctcctgccttagcctcccgagcagctgggattacaggttcctgccaccatgcccgtctaatttgtgtacttttagtagagatggggtttcgccatattggccaggctggtcttgaactcctgacctcaggtgatctgcctgcctcgtccttccaaagtgctaggattacaggcgtgagccactgcacacagcctacaaaagtgattttacttcaaagtttgaagtctgaagttttcctcttcattctaaattctctgtccttcttgggaatgattgcaacctcttcataagagtatgactccaaataacattgccacatcaaaagcctctgaggtcaaaatcaatacatgctggttccctccttcccttctccctttcctccttcccttcctccctctctccttccatccatccatccctccttttcttccatccctcagtatttgattctctactatgtgccacataatatagtagtgaaatagacagatgttgccctcatcctcacagagtttacatctagtggagaaacagaaaaaaacaaagtaattatggttcattgtgatgaatgtaatgacaaaggtaagctcaggatattatgagagtacacaggacgaatatctaaccccaacttggtagtcacggtggttttcttataagaagttacatctcagctgacaccaaaggataaataggagttttctgggcaaagaagtgtgtggatgggtagagttggaggaaggtgggaataagaggaaagagtggtcaagaatgagagaataccatgtgtaagatttggaggcaagaagtcacagtgccttggagaagctgtgtccatatggagcacatccttccattgtaatctcacttcctgagcttcctcttctactccctagtatccttggctttgtccatgggtcctaatttcaatccttatctttaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtaaagaaaaagtcacccagaagaatataagctttctttaaagtgtaaaccctttttcttactctactctaattcttaatgggagtttgggcaaggcaaagctggtggaagactttctaccatctcaattgtacttagctgtctctgagttcccttttcatcttctgatgactcttagggtttatatatactcctctgcattcatgttagagggaagagcctgactggggaaggaagggctaccccagagatgctgcacataattcttccatcttgctctgctgggctgccctaagaacagggatttggggccctgcttctccattatggcaatctaaaccaaccacagagagcagtcgcatgagtgatccaaatttcagcctgagccttggcaatcaccccgctaccaccaggtgggagggctccgtgtggacagtgtcctcagtcctgccagaagtcacaggttgggtgggtggacttgacagtgtatatgatttattcactcatttgttttgcattcaagttacaaataattgttttgaaaatatttcttacatatcttacttctctgagaacacagcagtgagttagggctcctgttctcatggatcttacattctacctggtagggtggtggagagacaggcaatacacaaagaagaaaaatatcagatcatgataagtattatacagagaattcaacagggtgatgagatagtgagtgagtgggtggttattttcaatgagttcagggaaggcctcttggggagtggacatttaagctgaactctgaatgatacaagcctgactgtaaaggagaaagccttgggaagggcagggaaagaacagcctgggaggtgggtacagtcaggcaagggctatgggacagaaacaagcttggaacaagcaaggatcagaaagaagatgggtgcggcaggagcagagtgggcaaagaggagagtggagggaggtgaatcccatcactgggcacctcaagttcttactaagtgacagccacaaagtagcagtgaggcctgatgtctgcagcaaacctgagccctctctcatggaagcgtatattgtttttcttttgcttttttcctggcaggtcaaaaatatgaagaataaagacaaacgtttaaagatcatgaatgagattcttagtggaatcaaggtgagaatctgagcgtaggtggctctctgtgggtaaggacagaagcagtggctctcttggcctttgcaaaccctggtagaggtgatggattcctgcttctgggccggttgtgtcacatgacttttgtttcatgtcctcctgtccctctcaggtcctccgttgaaccccaatttctatttattcaccctaaatagcttgttttgttttgacactttactgtagaagtgtttaaacatgtagaaaagtagagactatcatgaacccctacaaacttttcacccagctccaggaattatcagcatgacctaactagtttcatctataaacccacacccctcacacttgctagattattttaaagcaaatctcagagatcttgtcatttcattcataaatgctgtgtatgtagctctaaaagaaggatttattttaaaaatatagccacaataccagtatcatacctacaaaaaaaattaacattctttgataccatcaaagttctccagttgcctcatatgtgccttttgtttgttttgctgttggttagattgaataaggacccaaaaaatgtccatgcatgatatttaattgatagtgattttaatctactacagtctctctctctctgtctgtgtgtgtgtgtgtgtgtgtgtgtgtgtgtgtgtcttggtcattccaaaaaaactaggttatttgtcctatttcctgtatttcctgtaaattgatagttagatctaaaggtttgattgatttatatttgatttgggggcgggagtacaaggttgctttcaagtggtgctgtgtgcttcccaaactgcagcgatggtaacttgagcgggcccaggtgctgtcagcctgatccatcccttacaaggtttcctgctagtctttcactagagcagtggtttgtgatgctggcagcacattagaatcacctgagaaatttgaagtttgtaaaacccactgaggctgggagcctacttcaaaccattgaaattggactctgggagtgaggcccagacatgtgtatttttttaagttccctagatgattctaatatgccaccaaggtgaaaaacactattgtaagaattttatgatcattgcctaggtctgttatttcattaggaattgcaaaagagtgaaatttttaaattctgttattcccttgacattgattagctggaattttatgaagaagaattatctttcatcaacttgttggttaccctaaaatgaagtttgtatgggatagacagaaaaaatgcttgcttctttccctctatttgccagctttcatgataatgcatttcatcctccagaggtgaccaataagttcttttaactaaatattgttttgaattaatacatcttaacattatcctaaacttcttaaaaatgaaacagagcccccttttaactccttgttaattctccacacagagctctgacatgtccttctgtgaatgtggcaaaactaaaagagagagctggttttgaatctcagctctaccactgggtagcttaacttgccaaaagtttggcaaggttcccttgcctctctcaacttgtttccttatagtttaaatgaagataataactctacaaatgtaaggtgactaactcaactcaatttgcccagaactttttaagtcttaagactggaaggcctgtgtcctaggcagccctttagtcccaggcaaactggaacagttagtcatactaaggttgttataaagtgagataatgtatttaaaatgctttgtacatagtaggttgtcaacaaatacaagctacctttctgtaatcatactttataatctggatttctattccccacattttggggactatatcttaaaacatgggtggatcagatacacctggtgccctttcaggggcaatcatgtgagctgtattttttttcaacttattagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtgagtagcagaggggtccatgggctgcgtattcacctggacctgcatcagcttcctgaagaggaagttcacttttgatagactagctggcatctctaggcctgaccgcaagatccaggggacttgtccctctcaccgccccatgccacttttcctcctttgtaccatacccccagctctagtttcaccttcgtgtactgtacgatggtctggctcccagagcaacttttcaggatgcacagcaagaatgtggtatcaggaaggattggcttaggaggctttcttgtcttctttgggaggtggtgtaggtctatagtggaaggaacactgatataggatccaggagacctgagctctggtcctagtaatccattaattgactttgtgaccttggagaagatatttcctcctctcctgggctcagttttctcatctgtaacatggggaggctggatgatccttaaggcgccttcaactctgatatatggtgttcctgaaacttagcacaatgctgcttggtcccttttaggaataaattgctcatgaccttgccctttccaggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggtaggtcggcattctcactgctaactccctgtctctggttaaaagccttggagtccagaatgactggctatcacatcccatgtctaacttttgctggtaaactttcaagagagagatctgtttggtctctggagaccaatggtttgaccttaataaaatcatgagttttctgagccccagtttatctaactataaaatgaggataatcataccaatctcatctacctcacagggagacaatcttgtgaaagcatttagtgagattattatgtttagtcctagtactgggaacatttcccagcccagtttctctcatatttctccaaggctcttgaggtgttgagatagcattaggcaaatatggcccaaagggagaagagagaccctccctttgcataaacccatgtgttggtcaaaccaaatgcaaattgagagcctcaagctatcagaaaatgagtctggagttcaggaaaccttggatggaccagagggaatacctgggttctgaaaaactgagcatagtcactgatatgcgacaagtaccagatctgcagaggaaggtcaccggacaggcagcagactgatgagaaggcctgaatgacaggagtgtggtgacttgcatttatcccctaagtgtctggcttcagagagctcagaccatgggaaagagaaggcctccctgtcctggagggtgtaaagaaaagctggcagatttggcaggtaaagaagcaacaggatctttttagatacaatttattgcataaatagcaaaagcaagatcagcaaaggtatcagctccctgcattcctggtcctcaggatgacactgaatcaaaaagggtcagatggtggctgggctcacacctgtaatcccaacactttggaaggctgaggcaggcatatcacctgaggttaggagtttgagaccagcctggccaacatggtgaaaccccatctctactaaagaaacaaaaattagccaggcatggtggcacacacctgtagtcccagctactcgagaggctgaggcacgagaatcacttgaacctgggaggtggaggttgcagtgagccaagattgcacccttacactccagcctgggcgacagagtgagactccatctcaaaacaaaacaaaacgaatcaaaacaaaagggtcagatggcgatgcaatgtgagcagtggggtactctgtttttgaggagctgatccatgcagcagttaagtggttctatagcctgcttctgtaccctaaggagggaacaggcagaaagcctaagacttcatcagagcctgggtggcaatgaaaaaccatctcatgacagcctctttggaagagagggaagtaggtgagaaacatccttgtagaagtgcctcgaaatcttctcaagttctcaagttccagggactgcagggtattctgccaacacttacctcacctgccatttaagcttcggctatctggcctatgtggaagtgtgcagggtccccctgatgctatagccagttcctctttagaggctcccaggagtggcttagttaggatgctgggtgggcatcagggcatagacttagttaaataagagaaagccaggggcctagttaccagaaccagatagaagttaattttttgtcttagaaggcaggaggtagtttctggaattagggccgaagactggagagagacagcagaagagttgaactccatgagtttgctcaaggactgaaatagagcatccacttccatctgtctatggtgggatagcttccggggctgagttcggtggagattaggagatgccagctgtggcaaatagaaaatcatggactaacgacaaagtcaaaatctctctgcttgtgctcgttactgactttaattcttgagatcctttgtgtccttctctaggccagtgtttccacagagcggctagagaagtacttgggaggggatgacttggacacatctgccattcgacatgactgcaattttggtaaataaatttggaagttgcttcccaaacttattcgcagtactggtgccagaattttgataatacaagagcttagtagcagcaaactgagagagagtgtggagatttgtcttgagcctatgcttgcataagatacaaactgttcttacttgaattatgtttacacttagagcagattgctcaactagattgctaagtaaagctgggccagctcttctgaggataagcagggaatcctgcaaaagatccagaaagcagaagtcaggatagagccaagtgtgtgggattgggaaggtagcctggagtgaaagaatactcagattctgaaggtttaaagattgggggccagccatggccagagtgggcataggactggaatccatagtggggaggatttatatatggaggtggaattccgcggatc

SEQ ID NO: 4 - DNA, 314 bp, Homo sapiens
ggcagacagtcacgtggggacctacattggactaaatgagggctttttctcaacagacaaagccatgcagttttctgaggcctcctttacctgggaacatgattcggaagccacagtccgagagtgagttgccttctttccatcctaatgttctttagcattctcactgcctggcctattctcagaaaattccatgtaatagaatgcatggggaaatgagctatgtgcatgtcttcggttagatactagtgcaggataaacccctggagagatgagctgaaagcaaaggttttcagccccttcccctgatactg

SEQ ID NO: 5 - DNA, 690 bp, Homo sapiens
ctgtagatcacacaaaccctgaactggctggtgaggatgacatcagaaatagagaccaacattgtggctgttgagcgaataactgagtacacaaaagtggaaaatgaggtaaggaggaactggaaaaatccaggaacaaggcaaaaacaacatgcaactccttcgagagtgcatctttagaatttctccatattttacatgggcccataatgggtccctaaagtttcctttgctctaactcaaaggacctaatttcttcaaacttagaataggaaatggtttaggggatgtcgggtcttgcaacataccgaggagatctacagcacaagctcatggtgtatttttctttaactgaataaggtaggaactttccaaaagtgcaagtctagttcagcctattaaatgcagaggccattaggtagctgggacactgctacccttctcctgttctatgacacgagtcctgggtggactgttcggctgagttgccaccttataaaacttacttctcatcttgtctccttgccaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaaggtaggtggagtgaagga

SEQ ID NO: 6 - DNA, 355 bp, Homo sapiens
tccatctcaggccagtcctatccaccatctcccagtcctgaggaactccgaggtccttttctggcatgagccccaacagcccccttgtcctttcacttgcaggaccccatcctgttctctggaagcctgaggatgaatctcgaccctttcaacaactactcagatgaggagatttggaaggccttggagctggctcacctcaagtcttttgtggccagcctgcaacttgggttatcccacgaagtgaccagaggctggtggcaacctgaggtaatgttccatagcctgctacccctgcaggcaatgagaggatgtgagggggccactcctcgtgttcctcgtgttaggtgatgcc

SEQ ID NO: 7 - protein, 1545 aa, Homo sapiens [sequence omitted]